(19) United States
(12) Patent Application Publication
(10) Pub. No.: US 2021/0137923 A1
(43) Pub. Date: May 13, 2021

(54) TREATMENT OF SEXUAL DYSFUNCTION

(71) Applicant: Olive Therapeutics, LLC, Belmont, MA (US)

(72) Inventor: E. Quattrocki Knight, Belmont, MA (US)

(21) Appl. No.: 17/104,402

(22) Filed: Nov. 25, 2020

Related U.S. Application Data
(63) Continuation of application No. 15/969,471, filed on May 2, 2018, now abandoned.
Application No. 15/969,471 is a continuation-in-part of application No. 15/864,698, filed on Jan. 8, 2018.
(60) Provisional application No. 62/467,328, filed on Mar. 6, 2017.
(60) Provisional application No. 62/465,592, filed on Mar. 1, 2017.
(60) Provisional application No. 62/443,731, filed on Jan. 8, 2017.

(51) Int. Cl. A61K 31/506; A61K 9/00; A61P 15/12; A61K 31/137

(52) U.S. Cl. CPC A61K 31/506; A61K 31/137; A61P 15/12; A61K 9/0034

(57) ABSTRACT

The present invention describes novel methods for the treatment of sexual dysfunction.

### CROSS-REFERENCE TO RELATED APPLICATIONS

This application is a continuation-in-part of U.S. application Ser. No. 15/864,698 filed Jan. 8, 2018, which claims the benefit of priority of U.S. Provisional Application No. 62/443,731, filed Jan. 8, 2017, U.S. Provisional Application No. 62/465,592, filed Mar. 1, 2017, and U.S. Provisional Application No. 62/467,328, filed Mar. 6, 2017, all of which is incorporated herein by reference in their entirety.

### BACKGROUND

Sexual dysfunction can affect both men and women, with significant negative impact(s) on their quality of life and enjoyment.

### SUMMARY

The present disclosure provides an insight that trans-vaginal/trans-mucosal/topical treatment on or around the genital mucosa either alone or in combination with certain other therapeutic agents may be particularly effective for the treatment of sexual dysfunction.

The present disclosure proposes that aspects of sexual function in humans can be attributed to the activation of central pattern generators located within the spinal cords of both males and females. Although higher-level (cortical and subcortical) central nervous system mechanisms provide inhibitory influences, once activated, the spinal cord generators can control copulatory responses, motor behavior, and ultimately orgasm. As such, topical (on the scrotum, glans penis or vulva) or trans-vaginal treatment applied directly to the mucosa of the genitalia, could potentially access the peripheral nerves that participate in the central pattern generator circuit, and activate them accordingly. This disclosure proposes that local medication effects on the spinal reflexes/central pattern generators (CPGs) responsible for sexual behavior, arousal, and orgasm will provide benefit for sexual dysfunction without necessarily the need to directly influence higher-level central nervous system activity.

### BRIEF DESCRIPTION OF THE DRAWINGS

FIG. 1. Current medication for female sexual dysfunction (flibanserin) and for SSRI induced sexual dysfunction (buspirone and bupropion) target central mechanisms in the brain to stimulate dopamine (DA) and norepinephrine (NE) while concomitantly reducing serotonin (5HT) to enhance sexual behavior. In the current model, the balance between these three neurotransmitters, if shifted in favor of stimulation (reduced serotonin, increased NE and DA), will reach a tipping point and thereby encourage the sexual response. The influence of spinal mechanisms is largely ignored in this model.

FIG. 2. This illustration depicts the action of phosphodiesterase inhibitors for the treatment of male erectile dysfunction. Viagra®, other phosphodiesterase inhibitors, and most medications currently available for male erectile dysfunction target tissue mediated mechanisms to increase blood flow and thereby facilitate penile engorgement. The inhibition of phosphodiesterase in the corpus cavernosum raises nitric oxide levels, increasing the amount of soluble cGMP, and thereby inducing an erection through the enhancement of blood flow through the capillary bed of the corpus cavernosum. Alternatives to this category of medication also largely aim to enhance tissue engorgement by targeting the capillary bed of the corpus cavernosum. No current therapy for male sexual dysfunction targets spinal mechanisms.

FIG. 3. This diagram describes the basic building blocks of central pattern generators (CPGs). CPGs provide the neuronal impulses to induce rhythmic, coordinated muscle contractions that underlie important biological functions, including breathing and locomotion. In primitive species, CPGs orchestrate egg laying behavior (Weiss et al., 1992) and feeding (Jing et al., 2007). Given the pervasive and primitive nature of sexual behavior, CPGs most likely help coordinate the sexual response as well, even in humans. Panel A represents a building block of a CPG, the oscillating neuron capable of action potential bursts. Panel B depicts the simple two cell half-centered oscillator. In this manifestation, both neurons are capable of firing bursts of action potentials, but the activity in one neuron inhibits the activity in the opposing neuron. The opposing inhibition between the two cells allows for an alternating pattern of rhythmicity. This type of oscillating activity gives rise to locomotion and other behaviors that require alternating muscle contraction with opposing muscle relaxation. Panel C describes another type of CPG that produces rhythmic output. In this circuit, sensory input stimulates a motor output, but the delayed negative feedback loop transiently inhibits the transmission of the stimulatory signal, giving rise to rhythmic motor output. The muscle contractions associated with orgasm exemplify such an automatic rhythmic motor output response.

FIG. 4. This disclosure proposes that more direct modulation of spinal processes would have a beneficial effect on sexual function. Although higher level, central nervous system (cortical and subcortical) inhibitory control significantly influences sexual behavior, the present disclosure for treating sexual dysfunction appreciates the importance of spinal reflexes and the underlying central pattern generators transmitted through the spinal cord that mediate the sexual response, particularly orgasm. Topical, transmucosal, and/or transdermal applications of medications in the region of the genitalia could access the circuitry responsible for the sexual response and thereby either facilitate the response, in cases of reduced sexual function, or inhibit the response in cases of premature ejaculation and persistent genital disorder. In this diagram, the darker lines depict sensory (affective) neurons providing stimulation from the pelvic region back to the spinal cord circuit. The interneuron (lighter, short line within spinal cord) within the spinal dorsal horn of the spinal column transmits these impulses via the effector motor neurons to the anatomical organs and muscles of the pelvis. At the time of orgasm, the interneurons, by providing a delayed inhibition, potentially contribute to the coordinated rhythmicity of climax. This disclosure suggests that human spinal nerves innervating the pelvic region in both males and females resemble the CPG motif from panel C in FIG. 3 and may significantly contribute to the sexual response. Animal studies indicate that certain receptors, including, but not limited to, serotonin (5HT1A, SHT2A, and 5HT8), nicotinic, alpha-1 noradrenergic, dopamine, glutamate, GABA, melanocortin, oxytocin, melatonin, (Reiter et al., 2009), and vasoactive intestinal peptide receptors all contribute to the transmission of signals to and from the pelvic organs and the spinal cord. Modulation of this potential circuit via local application of medications (arrow) to act via the receptors expressed on the neurons transmitting both sensory input to the spinal cord and motor signals back from the spinal cord to pelvic regions could potentially treat sexual dysfunction.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Administration: As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be topical. In some particular embodiments, administration may be dermal (which may be or comprise, for example, one or more of topical to the dermis, intradermal, interdermal, transdermal, etc), mucosal, topical, or vaginal. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid. saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form.

Agonist: Those skilled in the art will appreciate that the term “agonist” may be used to refer to an agent condition, or event whose presence, level, degree, type, or form correlates with increased level or activity of another agent (i.e., the agonized agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered).

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Modulator of spinal reflex are: As used herein, the term “modulator of spinal reflex arc” refers to any agent capable of activation of central pattern generators, either through augmenting the signal transduction of affective (sensory) pathways traveling towards the spinal cord or enhancing the transduction of effective (motor) pathways traveling away from the spinal cord, either at the level of the synapses, ganglia, dorsal root horn, sensory nerve terminals, or muscle membranes of both males and females. In some embodiments, modulators of spinal reflex arcs may comprise potassium channel blockers, potassium channel openers, two-pore potassium channel modifiers, agents that act on ion co-transporters or exchangers, sodium channel openers, calcium channel openers, or acid sensing and proton channel blockers. In some embodiments, modulators of spinal reflex arcs may comprise oxytocin receptor agonists, serotonin receptor 1A (5HT-1A) agonists, serotonin 2C agonists, nitric oxide enhancers, cGMP elevators, PDE5 inhibitors, sympathomimetic agents, dopaminergic agents, glutamatergic agents, vasoactive intestinal peptide analogs or enhancers, melanocortin receptor analogs or enhancers, nicotinic receptor agents, melatonin or melatonin analogs, or buproprion.

Channel modifier: As used herein, the term “channel modifier” refers to any agent capable of inducing a response from a cellular channel. In some embodiments channel modifiers may comprise potassium channel blockers, potassium channel openers, two-pore potassium channel modifiers, sodium channel openers, calcium channel openers, or acid sensing and proton channel blockers.

### DETAILED DESCRIPTION OF CERTAIN EMBODIMENTS

### Central Pattern Generators

Central pattern generators (CPGs) are defined as a sequence or collection of neural cells that act as a wiring circuit that can drive repetitive rhythmical movement in a self-perpetuating fashion (Grillner et at, 2005; Grillner and Wallen, 1985). CPGs were first identified in invertebrates and have only more recently been recognized in higher species, including mammals. Most of the work on CPGs and sexual activity still focuses on drosophila (von Philipsborn et al., 2011; Yamamoto and Koganezawa, 2013), insects (Lange, 2009), and non-mammalian species (Leininger and Kelley, 2015). However, CPGs exist in humans. The collection of neurons in the brainstem that control breathing are an example of a CPG in humans. A recent report proposed that a CPG-like organization of neurons residing in the lumbosacral spinal cord controls male ejaculation in the rat (Carro-Juarez and Rodriguez-Manzo, 2008), suggesting that the same type of CPG may exist and control male ejaculation in the human male. To our knowledge, however, no scientist has proposed that CPGs control any other aspect of sexual behavior (arousal, foreplay, or intromissions) in the male. Nor was the finding of a CPG underlying ejaculation in the male suggested as a rationale for using topical or transmucosal applications of pharmacological treatment for ejaculatory dysfunction.

Although recently scientists have begun to recognize that CPGs control the pulsatile nature of hormone release in human females (Israel et al., 2014), an ejaculation response similar to males in female rats (Carro-Juarez and Rodriguez-Manzo, 2006), and the hind limb receptive posture in the cat (Kirkwood and Ford, 2004), no scientist has proposed that a spinal central pattern generator controls human female sexual behavior (desire, arousal, foreplay, or orgasm). Moreover, the ejaculatory response observed in female rats could only be elicited in spinally transected rats (Carro-Juarez and Rodriguez-Manzo, 2008).

The present disclosure offers the insight that if peripheral sensory and motor nerves and spinal reflexes might be involved in triggering the CPG responsible for aspects of the human sexual response, that local application of drugs could potentially induce, augment and/or facilitate the sexual response in humans. The authors who first identified the CPG responsible for ejaculation in rats suggested that their insight could be used to understand premature ejaculation in humans, but they did not propose any specific medication or the prospect of applying local (topical, transvaginal or trans-mucosal) medications as a possible therapeutic route or means to do so (Carro-Juarez and Rodriguez-Manzo, 2008). In their evaluation of the ejaculatory response in both male and female rats, the authors applied their pharmacological manipulation only through the intravenous route and never attempted topical, transmucosal, or transvaginal administration (Carro-Juarez et at, 2006; Carro-Juarez and Rodriguez-Manzo, 2005, 2008).

### Sexual Dysfunction

For purposes of present disclosure, sexual dysfunction includes, without limitation, impairment in sexual desire, arousal, orgasm, premature ejaculation, or satisfaction, any or all of which may be due to, for example, psychogenic, biologic (including, but not limited to: vasogenic, endocrine related, menopause, and neurologic disorders), or medication-induced mechanisms. Sexual dysfunction, as used herein, does not include pain or sexual paraphilias.

Sexual dysfunction can sometimes arise after treatment with particular therapeutic agents. For example, sexual dysfunction is a known side effect of therapy with certain selective serotonin re-uptake inhibitors, certain selective norepinephrine and serotonin re-uptake inhibitors, certain anti-hypertensive medications (e.g., alpha-2 adreno-receptor antagonists, beta-blockers, etc), certain antipshycotics D2 antagonists), etc. Such treatment emergent sexual dysfunction (TESD) affects between 26-80% of individuals taking antidepressant medication (Serretti et al., 2011) and significantly contributes to patient non-compliance (Monteiro et al., 1987; Segraves et al., 2014; Kennedy et al., 2009), with one study reporting that up to 90% of patients who develop TESD will prematurely discontinue their antidepressant medication (Nurnberg et al., 2009).

TESD adversely impacts quality of life, puts stress on relationships and markedly increases the risk of discontinuing antidepressant medication despite successful treatment response. Of the various sexual symptoms associated with TESD, orgasmic dysfunction (delayed, diminished or absent orgasm) is among the most common and the most clearly associated with the use of antidepressant medication (Labbate et al., 1998; Clayton et al., 2006). A prospective trial reported that delayed, or diminished orgasm occurred in over 60% of patients, and anorgasmia was reported in over 30% (Montejo, 2015). Although depressed patients often endorse low desire as well as difficulty achieving orgasm, low desire often predates antidepressant treatment and correlates with a depressed mood; whereas, delayed, diminished, or absent orgasm (anorgasmia) more likely emerges after the onset of treatment and frequently corresponds to TESD (Montgomery et al., 2002).

The exact mechanism of TESD, and in particular, antidepressant associated orgasmic disorder (ADOA), has not yet been clearly established, but appears to involve the serotonergic system and relates, in part, to the mechanism underlying disease amelioration. One study reports that the early emergence of delayed orgasm can actually predict a patient's likelihood of responding to SSRI treatment (Humble et al., 2016). Given this association, physicians often resist switching antidepressants on the basis of TESD alone. Furthermore, because the therapeutic benefits of antidepressant medications have a delayed onset, the cost of substituting a different antidepressant medication can involve a washout period (1-2 weeks) as well as the time necessary to properly evaluate the therapeutic potential of the new agent (4-6 weeks).

Current treatment of TESD includes: drug holidays, timing of coitus, dose reduction and the use of other oral agents that might act as antidotes (phosphodiesterase inhibitors, buspirone, bupropion, yohimbine). Little data exists to support any of these strategies or antidotes (Taylor et al., 2013). Drug holidays (i.e., skipping doses on the weekends) tends not to work for antidepressants with long half-lives (fluoxetine, Prozac®); whereas, stopping an antidepressant with a short half-life (paroxetine, Paxil®) increases the risk of the unpleasant symptoms associated with selective serotonin re-uptake (SSRI) discontinuation syndrome. A Chochrane Database review of the treatment for TESD reports that the evidence to date does not support the use of phosphodiesterase inhibitors to treat medication induced orgasm difficulties in either men or women and only weakly supports the use of high dose bupropion to help correct TESD in women (Taylor et al., 2013). The addition of high dose bupropion, however, increases the risk for other side effects, including seizures (Alper el al., 2007) and cognitive disturbances (Balon et al., 1996). Moreover, patients often resist adding yet another daily oral medication to their drug regimen, particularly if they already experience adverse consequences from their current treatment.

Women have a 33% possibility of developing depression over the course of their lives (Martin et al., 2013). The post-partum and child rearing years pose considerable risk for the appearance of depressive symptoms, and the burden of this disease perpetuates adverse ramifications through the generations. Maternal depression increases the incidence of infant and childhood negative outcomes, affecting everything from infant mortality to intelligence and adult resilience in the offspring (Surkan et al., 2016). Yet, sexual dysfunction can also amplify a woman's stress during this stage of life, predicting that the emergence of antidepressant related sexual dysfunction would compel a significant number of women to discontinue antidepressant medication. A safe and effective antidote for anti-depressant treatment emergent sexual dysfunction in pre-menopausal women could potentially increase compliance with antidepressant medication, ease the burden of depressive symptoms, and help protect numerous children from the adverse consequences of maternal depression. Treatment emergent sexual dysfunction remains a prevalent problem, with serious adverse consequences due to the high burden depression inflicts on society, and no well-established safe and effective treatment.

Current medication strategies for treatment of sexual dysfunction in women are designed to target known underlying medical causes, such as thyroid hormone for hypothyroidism and hormone replacement for menopause. The only currently approved medication for premenopausal women without a clear underlying medical cause, Flibanserin (trade name Addyi®), is designed to increase the frequency of satisfying sexual encounters. The exact mechanism of the drug is incompletely understood, but is believed to involve increases in norepinephrine and dopamine levels, coupled with a simultaneous decrease in serotonin release. See FIG. 1. Flibanserin is reported to act as an agonist to the serotonin 1A (5HT-1A) receptor, as a weak antagonist to the 5-HT2A receptor, and may show weak partial antagonism to the dopamine D4 receptor. The FDA recently approved this treatment (specifically, 100 mg pill taken orally at bedtime) for female hypoactive sexual interest/arousal dysfunction (FSIAD), but modest efficacy, the requirement to abstain from alcohol and the necessity of daily dosing, regardless of sexual frequency, have prevented this medication from becoming widely used. Furthermore, black box contraindications including (in addition to alcohol use), liver impairment, and concurrent use with other hepatic enzyme CYP3/4A inhibitors, combined with a risk of syncope and some concern about increased breast cancer risk have further discouraged its adoption. Moreover, flibanserin is only indicated for premenopausal women, despite the high prevalence of hypoactive sexual desire disorder in post-menopausal women.

In males, current therapies have more successfully mitigated sexual dysfunction, but have primarily been designed to enhance erections, not facilitate orgasm or mitigate premature ejaculation. The typical approach inhibits the phosphodiesterase enzyme (PDE5) from degrading cyclic guanosine monophosphate (cGMP) after nitric oxide has bound with soluble guanylate cyclase. See FIG. 2. The cGMP causes vasodilation (by relaxing smooth muscle) of the corpus cavernosum capillary bed, leading to subsequent tissue engorgement (erection). Typical drugs in this class include sildenafil (Viagra®), tadalafil (Cialis®), and vardenafil (Levitra®). Certain PDE5 inhibitor agents are marketed for oral administration (specifically, Viagra®, Levitra®, Cialis®, are typically prescribed in 25-100 mg doses taken by mouth approximately 4 hours to 30 minutes prior to engagement, as needed, but not more than once per day); alternatively or additionally, certain PDE5 inhibitor agents are marketed for sublingual and/or buccal administration. Clinical trials by Pfizer indicate that 63-82% of men with erectile dysfunction experience improved erectile dysfunction on Viagra®).

Other treatments for male erectile dysfunction also attempt to promote tissue engorgement through smooth muscle relaxation and subsequent vasodilation, but are either less effective than the phosphodiesterase inhibitors or require direct injection into the corpus cavernosum at the base of the penis. For example, Trimix® (a combination of phentolamine, PGE1, papivarine, administered in 0025-0.5 microgram doses), histamine (administered in 30-60 microgram doses (Cara et al., 1995)), and Invicorp® (a combination of 25 micrograms of Vasoactive Intestinal Peptide (VIP), an alpha-adrenergic antagonist that occludes venal outflow from the corpus cavernosum of the penis, with 1-2 mg of phentolamine mesylate, an alpha-adrenergic antagonist that increases blood flow thereto so that the combination allows for tissue engorgement (Dinsmore and Wyllie, 2008)).

Vasodilators (Prostaglandins, alpha-adrenergic blockers, histamine) and antispasmodic drugs (opium alkaloids) have been used alone and in combination with some success.

However, hypotension and headache are fairly common side effects of many of these modalities. Post marketing reports of vision problems have been distributed by the FDA. Men currently using nitrates for cardiac issues, men with cardiac output deficiencies, orthostatic hypotension, or hypovolemia should not PDE5 inhibitors.

Current treatment for premature ejaculation is primarily cognitive restructuring and couple's therapy. The available medical treatment includes oral administration of serotonin re-uptake inhibitors. Given their known effect on delaying orgasm, SSRI's have successfully treated premature ejaculation in some instances. The need for constant daily dosing and other side effects limit the usefulness of this approach. Topical treatments have also been prescribed with some success, but they have largely tried to delay orgasm by reducing sensation with numbing agents such as lidocaine and/or other topical anesthetics. No current approaches target the spinal reflexes or CPG's involved in orgasm.

As the most diverse of the ion channel families, several different potassium channel subtypes critically contribute to the rhythmicity, firing strength, and synchronization of excitable cells, and in particular, help orchestrate the excitability that underlies sexual excitement and orgasm. The smooth muscle, striated muscle, vasculature and nerves involved in the sexual response each express many different subtypes of potassium channels. When open, potassium channels passively allow for the flux of potassium ions with the direction of the potassium concentration gradient. Under normal physiological conditions, the potassium concentration gradient (higher internal than external) favors the flux of positively charged potassium ions out of the cell, re-establishing a negative membrane potential. Local environmental triggers, such as changes in voltage, more availability of adenosine triphosphate (ATP), proton levels, and calcium concentration, can all initiate the conformational change that opens particular potassium channels, depending on the specific subtype.

Because potassium channels primarily exert membrane stabilizing effects through the outward flux of positive ions (hyperpolarizing influence), potassium channel blockers, in general, promote membrane excitability by raising the internal membrane voltage and thereby lowering the threshold for action potential generation. Potassium channel blockers can also promote membrane excitability by broadening action potentials. The direct modulation of ion channels could harness the role these transmembrane proteins play in the excitability of the muscles and peripheral nerves innervating the genitalia that gives rise to the sexual response. Topical application of channel modifiers could thereby help alleviate sexual dysfunction. Without wishing to be bound by any particular theory, it is believed that enhancing the excitability of the nerves and muscles involved in the sexual response can promote the positive feedback loop necessary for sexual responses, and can lower the threshold for the onset of the central pattern generators involved in both generating arousal and facilitating orgasm.

Although potassium channels predominantly exert stabilizing influence on membrane potentials, agents that enhance potassium channel opening can paradoxically increase a cell's excitability (Quattrocki, Marshall et al. 1994). By hyperpolarizing the internal milieu, potassium channel activity can remove sodium and calcium channel inactivation. Because the activation of voltage gated sodium and calcium channels mediate the excitable response, removing the inactivation of these depolarizing ion channels helps coordinate action potential generation by promoting synchronous openings and could help favor the initiation of the rhythmicity underlying orgasm. The coordinated rhythmicity can also enhance the strength of muscle contractions.

A relatively newer family of potassium channels, the K2P channels (TREK, TRAAK, TWIK, TALK, TASK, THIK, TRIK) respond to the mechano stimulation of stretch. Although these channels have not been previously directly implicated in the sexual response, without wishing to be bound by any particular theory, because cervical stretch can stimulate sexual arousal in mammals and penile stretch also participates in mammalian sexual arousal, these ion channels may play a role in the affective signaling to the spinal cord that helps initiate the central pattern generators instrumental to sexual behavior and orgasm. Fluoxetine and its metabolite, nor-fluoxetine both inhibit K2P channels (Dong, Pike et al. 2015) and the inhibition of these channels may contribute to the sexual side effects associated with this antidepressant medication. Topical modifiers of these K2P channels may serve to facilitate the sexual response and to combat the sexual side effects of anti-depressant medications.

The potassium dependent sodium-calcium exchanger, under normal physiological conditions, harnesses the potential energy of the potassium and sodium concentration gradients to remove calcium from the cell. These membrane spanning proteins are expressed in many excitable cells, including the nerves of the peripheral nervous system and the smooth muscles of the viscera. Sodium-calcium co-transporters can operate in the “reverse-mode,” pulling calcium into the cell instead of extruding it (Lytton, Li et al. 2002). Intake of calcium ions strengthens the force of muscle contractions and can lower the action potential threshold. Vaginal secretions have a characteristic ionic composition that differs from serum plasma (Levin and Wagner 1977). Instead of high sodium and low potassium, vaginal secretions maintain a relatively low sodium concentration and high potassium content in the extracellular environment, but under conditions of sexual arousal, the concentration of sodium ions diminishes, a configuration that could reflect the uptake of calcium ions into the smooth muscles, nerves, and vasculature of the genitalia via the sodium-calcium, or potassium dependent sodium calcium exchangers. The topical application of medications or extracellular matrix gels that could facilitate the uptake of calcium into the excitable cells (nerves and muscles) in the reproductive tracts (both male and female) via the sodium-calcium or potassium dependent sodium calcium exchangers could thereby facilitate the sexual response.

Sodium flux into excitable cells generates membrane action potentials. Any local application of a sodium channel opener could potentially help augment the excitability of the tissues and central pattern generators involved in the sexual response.

Calcium influx stimulates contractions in muscle cells. The targeted application of a medication that enhances calcium channel opening could increase the strength of the orgasmic response.

Because the female vagina hosts an environment with a low pH under physiological conditions, acid and proton sensing channels, which normally permit sodium to enter the cell, would most likely be constituently active. The permissive entry of sodium into the cell under these conditions would inactivate many of the voltage-sensing ion channels, paradoxically diminishing the excitability of this tissue. Thus, the intravaginal use of medications that would block these channels, might help re-establish a hyperpolarized membrane potential that would release the inactivation of the sodium channels and could favor excitability, rhythmicity, and the activation of central pattern generators. Vaginal secretions become less acidic after sexual stimulation and during arousal (Levin, Wagner et at. 2001). Speculations about the nature of this pH change have only suggested that the rise in vaginal pH contributes to sperm viability and ultimately reproductive success, but this disclosure proposes that the pH increase during arousal may also serve to facilitate orgasm. Thus, the application of medications or gels that would facilitate the closing of acid sensing channels could facilitate sexual arousal and promote orgasm.

### Tropical, Trans-Mucosal, Transvaginal Therapy

As indicated above, the present disclosure provides the insight that local applications of one or more therapeutic agents, e.g., as described herein, can be particularly useful and/or effective in the treatment of sexual dysfunction. As described herein, where provided methods of treatment comprise multiple therapeutic agents, such multiple agents may be administered in any manner, for example simultaneously, concomitantly, or sequentially. In some embodiments, a provided method comprises a step of administering a therapeutic agent to a subject that is receiving or previously received treatment with one or more other therapeutic agents described herein for the treatment of sexual dysfunction.

Provided technologies for treating sexual dysfunction as described herein, may be particularly useful or effective in patient populations such as, but not restricted to:
1. Individuals with delayed orgasm or difficulty achieving orgasm either as a primary complaint or secondary to psychotropic medications such as the SSRI's.
2. Post-menopausal woman (the only current treatment available for woman, Flibanserin, is restricted to premenopausal women).
3. Individuals who do not adequately respond to available medications or are unable to tolerate available medications because of side effects or contraindications.
4. Males with hypoactive sexual desire.
5. Males with premature ejaculation.
6. Females and males with persistent genital arousal disorder (PGAD).

Dosing: In contrast to flibanserin dosing, which requires patients to take the medication on a daily basis regardless of whether they anticipate imminent sexual behavior, the local treatment provided by the present disclosure could be administered on an as needed basis, from approximately 30 minutes prior to anticipated engagement and could be incorporated into foreplay or other aspects of the sexual act.

Local application: Topical (transdermal, trans-mucosal), trans-vaginal, or intrarectal application can deliver a therapeutic agent (e.g., a water-soluble agent) to local neurons involved in the spinally controlled behavior via either local blood supply, diffusion, or even retrograde transport within neurons.

Representative exemplary agents: the present disclosure teaches that known mediators of the ejaculatory response in rats could potentially also benefit the sexual response in humans. Although all pharmacological agents used in the experiments to evaluate the ejaculatory spinal reflex in rats were only administered intravenously (Carro-Juárez et al., 2003; Carro-Juarez et al., 2006; Carro-Juarez and Rodriguez-Manzo, 2005), this disclosure proposes certain of these agents may also have therapeutic efficacy if administered locally as described herein. In some embodiments, such agents include: oxytocin, a serotonin 1A agonist, and nitroprusside.

In certain embodiments, agents (e.g., modulators of spinal reflex arc or channel modifiers) useful in accordance with provided methods include (either alone or in combination with other agents), the categories of agents listed below targeting receptors or pathways expressed by the neurons within the spinal circuits:
1. Oxytocin (syntocinon®) or other oxytocin receptor agonists
2. One or more serotonin 1A agonists (or partial agonists or serotonin 2C agonists): Flibanserin is one such agent, currently only used as an oral agent. Buspirone and Trazodone are other such agents. See infra for additional exemplary serotonin 1A (5HT-1A) agonists.
3. One or more compounds that augment the nitric oxide pathway, including arginine, cGMP elevators, and PDE5 inhibitors. Typical nitric oxide enhancers include sildenafil (Viagra®), tadalafil (Cialis°), vardenafil (Levitra®), 8-bromoguanosine 3′,5′-cyclic monophosphate, minoxidil, arginine, and nitroglycerin.
4. One or more sympathomimetic agents (primarily alpha-adrenergic agents) such as ephedrine, pseudoephedrine, or oxymetazolam. See infra for additional exemplary sympathomimetic agents.
5. One or more vasoactive intestinal peptide (VIP) analogues or enhancers (e.g., that may provide degradation inhibition by blocking neutral endopeptidase, NED, or soluble endopeptidase, SED, pathway); without wishing to be bound by any particular theory, the present disclosure proposes that, in some embodiments, such agents can augment VIP, a neuropeptide mentioned above that participates in the arousal response. Such agents include VIP and more stable VIP analogues (Igarashi, Ito of al. 2005).
6. Buproprion; in some embodiments, this medication can increase norepinephrine and/or dopamine levels in the synaptic cleft.
7. Neuropeptide Y inhibitors or neuropeptide Y receptor antagonists such as BIBP-3226, given that neuropeptide Y receptors modulate the autonomic nervous system and neurons within the pelvis and spinal cord express these receptors.
8. Melanocortin receptor agonists or enhancers, such as Bremelantoide, and agouti-related peptide (AgRP) inhibitors or AGRP blocking analogues such as vTv Therapeutic's agents HPP515, TTP2515, TTP515.
9. Melatonin or agents that activate the melatonin receptor such as agomelatine.
10. Agents that antagonize the serotonin 5HT-2A receptor, such as Trazodone®, hydroxazine, or cyproheptadine.
11. Dopaminergic agents such as cabergoline.
12. Glutamate antagonists or partial antagonists such as amantadine and memantine.
13. Vasopressin agonists such as vasopressin, lypressin, terlipressin, felypressin, and desmopressin.
14. Potassium channel blockers such as amiodarone, dofetilide, sotalol, ibutilide, azimilide, bretylium, clofilium, nifekalant, tedisamil, sematilide, sulfonylureas, and dalfampridine.
15. Potassium channel openers such as minoxidil, diazoxide, nicrandil, pinacidil, retigabine, and flupirtine
16. Two-pore potassium channel modifiers such as arachidonic acid, vernakalant, polyunsaturated fatty acids, and lysophospholipids.
17. Sodium channel openers such as solnatide.
18. Calcium channel openers such as ambroxol.
19. Acid sensing and proton channel blockers such as amiloride, amantadine, and rimantadine.
20. Ion co-transporter modifying agents such as gels with characteristic ionic compositions to facilitate calcium entry (calcium carbonate, low sodium, high potassium).

In certain embodiments, agents (e.g., modulators of spinal reflex arc or channel modifiers) useful for premature ejaculation in accordance with provided methods include (either alone or in combination with other agents):
1. Serotonin re-uptake inhibitors. Typical drugs in this class include fluoxetine, sertraline, and paroxetine.
2. Alpha-1 receptor antagonists or alpha 2 receptor agonists. Typical drugs in these two categories include clonidine and guanfasine.
3. Beta adrenergic receptors such as propranolol.
4. Agouti-related peptide, AGRP analogues, neuropeptide Y and/or neuropeptide Y analogues, and neuropeptide Y receptor agonists such as dexamethasone.
5. Melanocortin receptor antagonists.
6. Certain serotonin receptor antagonists such as Quetiapine® and Ketanserin®.
7. Vasopressin receptor antagonists such as Reclovatpin®.
8. Oxytocin receptor antagonists such as Retosiban®.
9. Nicotinic receptor antagonists such as dextromethorphan.
10. Serotonin 5HT-1A antagonists or 5HT-2A agonists.
11. Melatonin antagonists such as luzindole.
12. Prolactin or prolactin like non peptide agents.
13. Nicotinic receptor agents such as varenicline and cytisine. Neurons within the spinal circuits involved in arousal and orgasm express nicotinic receptors.
14. K2P channel blockers, such as topical fluoxetine.
15. Acid sensing channel openers.
16. Ion co-transporter or exchange modifiers such as ionic gels.

In certain embodiments, agents (e.g., modulators of spinal reflex arc or channel modifiers) useful for persistent genital arousal disorder, in accordance with provided methods include (either alone or in combination with other agents):
1. Serotonin re-uptake inhibitors. Typical drugs in this class include fluoxetine, sertraline, and paroxetine.
2. Alpha-1 receptor antagonists or alpha 2 receptor agonists. Typical drugs in these two categories include clonidine and guanfacine.
3. Beta adrenergic receptors such as propranolol.
4. Agouti-related peptide, AGRP analogues, neuropeptide Y and/or neuropeptide Y analogues, and neuropeptide Y receptor agonists such as dexamethasone.
5. Melanocortin receptor antagonists.
6. Certain serotonin receptor antagonists such as Quetiapine® and Ketanserin®.
7. Vasopressin receptor antagonists such as Reclovatpin®.
8. Oxytocin receptor antagonists such as Retosiban®.
9. Nicotinic receptor antagonists such as dextromethorphan.
10. Serotonin 5HT-1A antagonists or 5HT-2A agonists.
11. Melatonin antagonists such as luzindole.
12. Prolactin or prolactin like non peptide agents.
13. Nicotinic receptor agents such as varenicline and cytisine. Neurons within the spinal circuits involved in arousal and orgasm express nicotinic receptors.
14. K2P channel blockers such as topical fluoxetine.
15. Acid sensing channel openers.
16. Ion co-transporter or exchange modifiers such as ionic gels.
   5HT-1A agonists

The skilled artisan will be aware of a variety of 5HT-1A agonists, any of which may be used in accordance with the present invention. In some embodiments, a 5HT-1A agonist is selected the following nonlimiting examples: VPI-013 (Otsuka); Aripiprazole transdermal; RP 5063 (Reviva); Osemozotan; Vilazodone; Vortioxetine; Xaliproden; PF 217830, Zalospirone, Sunepiron and Sumanirole (Pfizer); Bifeprunoz; Gepirone; Pardoprunox; Lurasidone; Tandospirone; Sarisotan; Piromelatine; Eltoprazine; F17464, F15599, and Befiradol (Pierre Fabre); Naluzotan; TGBA01AD; TGFK 09SD and TGW 00AA (Fabre-Kramer Pharmaceutical); SKL PSYNZ 016 (SK biopharmaceuticals); Lisuride; Piclozotan; A74283 (Abbott); PSN 602 (Prosidion); Flesinoxan, SLV313, Umespirone and Elopiprazole (Solvay); LY301317, LY228729, LY293284, LY274601, LY274600, LY178210, and VML 670 (Lilly); SSR181507, RU24969, MDL73975, MDL72832, and Binospirone (Sanofi-Aventis); VN2222 and VN8522 (Vita); R 137696 (Johnson and Johnson); Buspirone transdermal, BMS184111, BMS181100, BMS181101, BMY7378, and BMY42568 (Bristol-Myers Squibb); EF7412, B20991 (CEPA Schwarz Pharma); LB 50016 (LG Chem investment); Nemonapride; Eptapirone; E5165 and Lesopitron (Esteve); HT90B (Chugai Pharmaceutical); WAY100012 (Wyeth); AP521, AP159 (Asahi Kasei); Repinotan (Bayer), S14671, S213571, S14506, Alnespirone, and S215521 (Servier); FG5893, FCE23892, U86170, U92016A, U93385, U67413B, and FG5865 (Pharmacia Corporation); SUN8399 (Suntory); EMD77697, EMD56551, Roxindole (Merck); LY41 (Uppsala University); RL315535 (Shire); Ebalzotan, PM1000 (AstraZeneca); LEK8804 (LEK); F92502CN, F8910RS (FAES Farma); Ipsapirone (Bayer); CGS18102, CGS12066B, CGP50281 (Novartis); SL870765 (Sanofi-Synthelabo); BP554 (Mitsubishi Chemical).

Partial agonists of 5HT-1A as well, such as Buspirone, are also useful in accordance with the provided methods.

### Sympathomimetic Agents

The skilled artisan will be aware of a variety of sympathomimetic agents, any of which may be used in accordance with the present invention. In some embodiments, a sympathomimetic agent is selected from among the following nonlimiting examples: phenylephrine, pseudoephedrine, armodafinil, modafinil, ephedrine, methoxamine, oxymetazoline, Arbutamine; ESR1150CL (Boehringer Ingelheims); NS49 (Nippon Shinyaku); A204176, ABT866 (Abbott); GW515524B (GlaxoSmithKline); Dabuzalgron (Roche); S19014, S172651 (Servier); SUNC5174 (Suntory); SL251039 (Sanofi-Synthelabo); RWJ152807 (Johnson and Johnson); SGB1534 (Chugai Pharmaceutical); and SDZNVI085 (Novartis).

### Dopaminergic Agents

The skilled artisan will be aware of a variety of dopaminergic agents, including dopamine receptor agonists, partial agonists, inverse agonists, and indirect agonists, any of which may be used in accordance with the present invention. In some embodiments, a dopaminergic agent is selected from among the following nonlimiting examples: amantadine, memantine, cabergoline, amphetamine, dextroamphetamine, buproprion, bromocriptine, apomorphine, ciladopa, dihexidrine, dinapsoline, doxanthrine, epicriptine, lisuride, pergolide, piribedil, pramipexole, propylnorapomorphine, quinagolide, ropinirole, rotigotine, roxindole, sumanirole, lisdexamfetamine, methylphenidate, p-tyramine, and phenethylamine.

### Glutamatergic Agents

The skilled artisan will be aware of a variety of glutamatergic agents, any one of which may be used in accordance with the present invention. In some embodiments, a glutamatergic is selected from among the following nonlimiting examples: NMDA receptor positive allosteric modulators (e.g., glycine, D-cycloserine and D-serine), glycine reuptake inhibitors (e.g. N-(3-(4′-fluorophenyl)-3-(4′-phenylphenoxy)propyl) sarcosine and glycyldodecylamide), glutamate reuptake inhibitor (e.g., excitatory amino-acid transporters EAAT3 antagonists), metabotropic glutamate receptors agonists (e.g., LY-354740), AMPA/kainate receptor antagonists (e.g., LY-293558, GYKI52466 and LY-326325), ampakines (CX-516), and glutathione prodrugs.

### Potassium Channel Blockers

The skilled artisan will be aware of a variety of potassium channel blockers, any one of which may be used in accordance with the present invention. In some embodiments, a potassium channel blocker is selected from the following nonlimiting examples: amiodarone, dofetilide, sotalol, ibutilide, azimilide, bretylium, clofilium, nifekalant, tedisamil, sematilide, sulfonylureas, and dalfampridine.

### Potassium Channel Openers

The skilled artisan will be aware of a variety of potassium channel openers, any one of which may be used in accordance with the present invention. In some embodiments, potassium channel opener is selected from the following nonlimiting examples: Minoxidil, diazoxide, nicrandil, pinacidil, retigabine, and flupirtine,

### Two-Pore Potassium Channels Modifiers

The skilled artisan will be aware of a variety of K2P channel modifiers, any one of which may be used in accordance with the present invention. In some embodiments, a K2P channel modifier is selected from the following nonlimiting examples: arachidonic acid, vernakalant, polyunsaturated fatty acids, lysophospholipids, and fluoxetine.

### Sodium Channel Openers

The skilled artisan will be aware of a variety of sodium channel openers, any one of which may be used in accordance with the present invention. In some embodiments, a sodium channel opener is solnatide.

### Calcium Channel Openers

The skilled artisan will be aware of a variety of calcium channel openers, any one of which may be used in accordance with the present invention. In some embodiments, a calcium channel opener is ambroxol.

### Acid Sensing and Proton Channel Blockers

The skilled artisan will be aware of a variety of acid and proton sensing channel blockers, any one of which may be used in accordance with the present invention. In some embodiments, an acid and proton sensing channel blocker is selected. from the following nonlimiting examples: amiloride, amantadine, and rimantadine.

Ion Co-Transporter and Exchange Modifiers

The skilled artisan will be aware of a variety of ion co-transporter and exchange modifiers, any one of which may be used in accordance with the present invention. In some embodiments, such a modifier is selected from the following nonlimiting examples: thiazides, aspartate, losartan, vaptans, and ionic gels.

### Formulations

The skilled artisan will be aware of a variety of formulations, any of which may be used in accordance with the present invention. In some embodiments, a formulation is a gel. In some embodiments, a formulation is a topical gel. In some embodiments, a formulation comprises phenyl ephrine. In some embodiments, a formulation comprises buspirone. In some embodiments, a formulation comprises phenylephrine and buspirone. In some embodiments, a formulation comprises about 0.05% phenylephrine and about 0.05% buspirone. In some embodiments, a formulation comprises about 0.05% to about 0.1% phenylephrine and about 0.05% to about 0.1% buspirone.

### EXEMPLIFICATION

### Example 1

A postmenopausal female subject not experiencing anorgasmia self-administered approximately 5 mL of topical gel comprising about 0.05% phenylephrine and about 0.05% buspirone prior to sexual activity. Active ingredients were provided as Buspirone in powder form together with dried guar gum and a liquid vial of phenylephrine in a normal saline base. The subject poured the liquid into ajar containing the powdered formulation and stirred until solidified, within an hour prior to sexual activity. Once the gel had been prepared, the subject applied the gel to external genitalia 5-10 minutes prior to engagement.

Upon completion of sexual activity, the subject self-reported the outcome of the qualities associated with the sexual experience based on the following criteria:
1. Pleasure (0-5 scale, where 1=reduced; 2=no change; 3=slight improvement; 4=noticeable improvement; 5=best ever)
2. Ease of orgasm (0-5 scale, where 1=reduced; 2=no change; 3=slight improvement; 4=noticeable improvement; 5=best ever)
   The results of trial are summarized below:
1. Pleasure
   Result: 5
1. Ease of Orgasm
   Result: 4

### Example 2

Subjects self-administer approximately 5 mL of topical gel comprising about 0.05% phenylephrine and about 0.05% buspirone prior to sexual activity. Active ingredients are provided as Buspirone in powder form together with dried guar gum and a liquid vial of phenylephrine in a normal saline base. The subjects pour the liquid into a vessel containing the powdered Buspirone and stir until solidified, prior to sexual activity. Once the gel is prepared, the subject applies the gel to external genitalia 5-10 minutes prior to engagement.

Upon completion of sexual activity, the subjects self-report the outcome of the qualities associated with the sexual experience based on the following criteria:
1. Pleasure (0-5 scale, where 1=reduced; 2=no change; 3=slight improvement; 4=noticeable improvement; 5=best ever)
2. Ease of orgasm (0-5 scale, where 1=reduced; 2=no change; 3=slight improvement; 4=noticeable improvement; 5=best ever)
3. Desire (0-5 scale, where 0=no interest and 5=self-initiated and more intense than ever experienced)
4. Arousal (0-5 scale, where 0=no lubrication or heightened sensitivity and 5 =most extreme arousal ever experienced)

### Example 3

Subjects self-administer approximately 5 mL, of topical gel comprising about 0.05% buspirone prior to sexual activity. Active ingredients are provided as Buspirone in powder form together with dried guar gum and a liquid vial of normal saline base. The subjects pour the liquid into a vessel containing the powdered Buspirone and stir until solidified, prior to sexual activity. Once the gel is prepared, the subject applies the gel to external genitalia 5-10 minutes prior to engagement.

Upon completion of sexual activity, the subjects self-report the outcome of the qualities associated with the sexual experience based on the following criteria:
1. Pleasure (0-5 scale, where 1=reduced; 2=no change; 3=slight improvement; 4=noticeable improvement; 5=best ever)
2. Ease of orgasm (0-5 scale, where 1=reduced; 2=no change; 3=slight improvement; 4=noticeable improvement; 5=best ever)
3. Desire (0-5 scale, where 0=no interest and 5=self-initiated and more intense than ever experienced)
4. Arousal (0-5 scale, where 0=no lubrication or heightened sensitivity and 5=most extreme arousal ever experienced)

### Example 4

Subjects self-administer a therapeutically effective amount of one or more modulators of spinal reflex arc prior to sexual activity. Active ingredients are provided as the modulator of spinal reflex arcs (optionally together with dried guar gum and a liquid vial of normal saline base). If combining dry and liquid ingredients, the subjects pour the liquid into a vessel containing the modulator of spinal processes and stir, prior to sexual activity. The subject applies the active ingredient(s) (optionally as a gel from the combination of dry and liquid ingredients) to external genitalia 5-10 minutes prior to engagement.

Upon completion of sexual activity, the subjects self-report the outcome of the qualities associated with the sexual experience based on the following criteria:
1. Pleasure (0-5 scale, where 1=reduced; 2=no change; 3=slight improvement; 4=noticeable improvement; 5=best ever)
2. Ease of orgasm (0-5 scale, where 1=reduced; 2=no change; 3=slight improvement; 4=noticeable improvement; 5=best ever)
3. Desire (0-5 scale, where 0=no interest and 5=self-initiated and more intense than ever experienced)
4. Arousal (0-5 scale, where 0=no lubrication or heightened sensitivity and 5=most extreme arousal ever experienced)

### Example 5

Subjects self-administer a therapeutically effective amount of one or more channel modifiers prior to sexual activity. Active ingredients are provided as the channel modifier (optionally together with dried guar gum and a liquid vial of normal saline base). If combining dry and liquid ingredients, the subjects pour the liquid into a vessel containing the channel modifier and stir, prior to sexual activity. The subject applies the active ingredient(s) (optionally as a gel from the combination of dry and liquid ingredients) to external genitalia 5-10 minutes prior to engagement.

Upon completion of sexual activity, the subjects self-report the outcome of the qualities associated with the sexual experience based on the following criteria:
1. Pleasure (0-5 scale, where 1=reduced; 2=no change; 3=slight improvement; 4=noticeable improvement; 5=best ever)
2. Ease of orgasm (0-5 scale, where 1=reduced; 2=no change; 3=slight improvement; 4=noticeable improvement; 5=best ever)
3. Desire (0-5 scale, where 0=no interest and 5=self-initiated and more intense than ever experienced)
4. Arousal (0-5 scale, where 0=no lubrication or heightened sensitivity and 5=most extreme arousal ever experienced)

### REFERENCES

Alper K., Schwartz K. A., Kolts R. L., Khan A. (2007). Seizure incidence in psychopharmacological clinical trials: an analysis of Food and Drug Administration (FDA) summary basis of approval reports. Biol Psychiatry. 62, 345-354.
Balon R. (1996). Bupropion and nightmares. Am J Psychiatry. 153, 579-580.
Cara, A. M., Lopes-Martins, Antunes, E., Nahoum, C. R., and De Nucci, G. (1995). The role of histamine in human penile erection. British journal of urology 75, 220-224.
Carro-Juárez, M., Cruz, S. L., and Rodriguez-Manzo, G. (2003). Evidence for the involvement of a spinal pattern generator in the control of the genital motor pattern of ejaculation. Brain research 975, 222-228.
Carro-Juarez, M., Lobaton, I., Benitez, O., and Espiritu, A. (2006). Pro-ejaculatory effect of the aqueous crude extract of cihuapatli (Montanoa tomentosa) in spinal male rats. J Ethnopharmacol 106, 111-116.
Carro-Juarez, M., and Rodriguez-Manzo, G. (2005). Evidence for the presence and functioning of the spinal generator for ejaculation in the neonatal male rat. International journal of impotence research 17, 270-276.
Carro-Juarez, M., and Rodriguez-Manzo, G. (2006). Evidence for the presence of the spinal pattern generator involved in the control of the genital ejaculatory pattern in the female rat. Brain research 1084, 54-60.
Carro-Juarez, M., and Rodriguez-Manzo, G. (2008). The spinal pattern generator for ejaculation. Brain Res Rev 58, 106-120.
Clayton A, Keller A, McGarvey E. L. (2006). Burden of phase-specific sexual dysfunction with SSRIs. Journal of affective disorders. 91, 27-32.
Dinsmore, W. W., and Wyllie, M. G. (2008). Vasoactive intestinal polypeptide/phentolamine for intracavernosal injection in erectile dysfunction. BJU international 102, 933-937.
Dong, Y. Y., A. C. Pike, A. Mackenzie, C. McClenaghan, P. Aryal, L. Dong, A. Quigley, M. Grieben, S. Goubin, S. Mukhopadhyay, G. F. Ruda, M. V. Clausen, L. Cao, P. E. Brennan, N. A. Burgess-Brown, M. S. Sansom, S. J. Tucker and E. P. Carpenter (2015). “K2P channel gating mechanisms revealed by structures of TREK-2 and a complex with Prozac.” Science 347(6227): 1256-1259.
Grinner, S., Markram, H., De Schutter, E., Silberberg, G., and LeBeau, F. E. N. (2005). Microcircuits in action—from CPGs to neocortex. Trends in neurosciences 28, 525-533.
Grillner, S., and Wallen, P. (1985). Central pattern generators for locomotion, with special reference to vertebrates. Annual review of neuroscience 8, 233-261.
Igarashi, H., T. Ito, S. A. Mantey, T. K. Pradhan, W. Hou, D. H. Coy and R. T. Jensen (2005). “Development of Simplified Vasoactive Intestinal Peptide Analogs with Receptor Selectivity and Stability for Human Vasoactive Intestinal Peptide/Pituitary Adenylate Cyclase-Activating Polypeptide Receptors.” Journal of Pharmacology and Experimental Therapeutics 315(1), 370.
Israel, J. M., Cabelguen, J. M., Le Masson, G., Oliet, S. H., and Ciofi, P. (2014). Neonatal testosterone suppresses a neuroendocrine pulse generator required for reproduction. Nature communications 5, 3285.
Jing, J., Vilim, F. S., Horn, C. C., Alexeeva, V., Hatcher, N. G., Sasaki, K., Yashina, I., Zhurov, Kupfermann, L, Sweedler, J. V., el at. (2007). From Hunger to Satiety: Reconfiguration of a Feeding Network by Aplysia Neuropeptide Y. The Journal of Neuroscience 27, 3490-3502.
Kennedy S. H., Rizvi S. (2009). Sexual dysfunction, depression, and the impact of antidepressants. Journal of clinical psychopharmacology. 29, 157-164.
Kirkwood, P. A., and Ford, T. W. (20)4). Do respiratory neurons control female receptive behavior: a suggested role for a medullary central pattern generator? Progress in brain research 143, 105-114.
Labbate L. A., Grimes J. B., Arana G. W. (1998). Serotonin reuptake antidepressant effects on sexual function in patients with anxiety disorders. Biol Psychiatry. 43, 904-907.
Lange, A. B. (2009). Neural mechanisms coordinating the female reproductive system in the locust. Frontiers in bioscience (Landmark edition) 14, 4401-4415.
Leininger, E. C., and Kelley, D. B. (2015). Evolution of Courtship Songs in Xenopus: Vocal Pattern Generation and Sound Production. Cytogenetic and genome research 145, 302-314.
Levin, R. J. and G. Wagner (1977). “Human vaginal fluid-ionic composition and modification by sexual arousal [proceedings].” J Physiol 266(1): 62P-63P.
Lytton, J., X. F. Li, H. Dong and A. Kraev (2002). “K+-dependent Na+/Ca2+ exchangers in the brain,” Ann N Y Acad Sci 976: 382-393.
Martin L. A., Neighbors H. W., Griffith D. M. (2013). The experience of symptoms of depression in men vs women: analysis of the National Comorbidity Survey Replication. JAMA Psychiatry. 70, 1100-1106.
Monteiro W. O., Noshirvani H. F., Marks I. M., Lelliott P. T. (1987). Anorgasmia from clomipramine in obsessive-compulsive disorder. A controlled trial. Br J Psychiatry. 151, 107-112.
Montejo A. L., Montejo L., Navarro-Cremades F. (2015). Sexual side-effects of antidepressant and antipsychotic drugs. Current opinion in psychiatry. 28, 418-423.
Nurnberg H. G. (2008). An evidence-based review updating the various treatment and management approaches to serotonin reuptake inhibitor-associated sexual dysfunction. Drugs of today (Barcelona, Spain: 1998). 44, 147-168.
Quattrocki, E. A., J. Marshall and L. K. Kaczmarek (1994). “A Shah potassium channel contributes to action potential broadening in peptidergic neurons.” Neuron 12(1): 73-86.
Reiter, R. J., Tan. D. X., Manchester, L. C., Paredes, S. D., Mayo, J. C., and Sainz, R. M. (2009). Melatonin and reproduction revisited. Biology of reproduction 81, 445-456.
Segraves Balon R. (2014). Antidepressant-induced sexual dysfunction in men. Pharmacology Biochemistry and Behavior. 121, 132-137.
Serretti A., and Chiesa A. (2011). Sexual side effects of pharmacological treatment of psychiatric diseases. Clin Pharmacol Ther. 89, 142-147.
Surkan P. J., Patel S. A., Rahman A. (2016). Preventing infant and child morbidity and mortality due to maternal depression. Best Pract Res Clin Obstet Gynaecol. 36, 156-168.
Taylor M. J., Rudkin L., Bullemor-Day P., Lubin J., Chukwujekwu C., Hawton K. (2013). Strategies for managing sexual dysfunction induced by antidepressant medication. The Cochrane database of systematic reviews. Cd003382.
von Philipsborn, A. C., Liu, T., Yu, J. Y., Masser, C., Bidaye, S. S., and Dickson, B. J. (2011). Neuronal control of Drosophila courtship song. Neuron 69, 509-522.
Weiss, K. R., Brezina, V., Cropper, E. C., Hooper, S. L., Miller, M. W., Probst, W. C., Vilim, F. S., and Kupfermann, I. (1992). Peptidergic co-transmission in Aplysia: functional implications for rhythmic behaviors. Experientia 48, 456-463.
Yamamoto, D., and Koganezawa, M. (2013). Genes and circuits of courtship behaviour in Drosophila males. Nature reviews Neuroscience 14, 681-692.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1.-31. (canceled)

32. A method of treating a subject suffering from or susceptible to sexual dysfunction comprising administering one or both of a modulator of spinal reflex arc and a channel modifier so that the subject receives treatment with both.

33. The method of claim 32, wherein the subject is suffering from or susceptible to a sexual dysfunction selected from delayed orgasm; difficulty achieving orgasm secondary to psychotropic medications; impairment in sexual desire, arousal, orgasm, or satisfaction, or a combination thereof.

34. The method of claim 32, wherein the subject is female.

35. The method of claim 32, wherein the subject is a post-menopausal woman.

36. The method of claim 32, wherein the subject is male.

37. The method of claim 32, wherein the sexual dysfunction is treatment emergent sexual dysfunction.

38. The method of claim 32, wherein the modulator of spinal reflex arc comprises oxytocin receptor agonists, serotonin receptor 1A (5HT-1A) agonists or 5HT-1A partial agonists.

39. The method of claim 38, wherein the modulator of spinal reflex arc is administered to the subject.

40. The method of claim 39, wherein administration of the modulator of spinal reflex arc is mucosal.

41. The method of claim 32, wherein the channel modifier comprises at least one potassium channel modifier, sodium channel modifier, calcium channel modifier, or acid sensing and proton channel modifier.

42. The method of claim 41, wherein the potassium channel modifier is a potassium channel blocker, a potassium channel opener and/or a two-pore potassium (K2P) channel modifier.

43. The method of claim 41, wherein the sodium channel modifier is a sodium channel opener.

44. The method of claim 41, wherein the calcium channel modifier is a calcium channel opener.

45. The method of claim 41, wherein the acid sensing and proton channel modifier is an acid sensing and proton channel blocker.

46. The method of claim 32, comprising administering at least one ion co-transporter or exchanger modifier.

47. The method of claim 46, wherein the at least one ion co-transporter or exchanger modifier comprises a modifier of sodium calcium exchanger or a potassium dependent sodium calcium exchanger.

48. The method of claim 41, wherein the channel modifier is administered to the subject.

49. The method of claim 48, where administration of the channel modifier is topical.

50. The method of claim 32, wherein the subject is receiving or previously received treatment with one or more of a serotonin re-uptake inhibitor, a selective norepinephrine and serotonin re-uptake inhibitor, an anti-hypertensive medication, an antipsychotics, or a combination thereof.

51. The method of claim 32, wherein the subject is receiving or previously received treatment with one or more other therapeutic agents for the treatment of sexual dysfunction.

52. The method of claim 32, wherein the method further comprises administration of at least one additional therapeutic agent effective for the treatment of sexual dysfunction.
